(19) United States
(12) Patent Application Publication
(10) Pub. No.: US 2025/0382584 A1
(43) Pub. Date: Dec. 18, 2025

(54) CELL

(71) Applicants: Koji TANABE, Palo Alto, CA (US); I Peace, Inc., Palo Alto, CA (US)

(72) Inventor: Koji TANABE, Palo Alto, CA (US)

(73) Assignees: Koji TANABE, Palo Alto, CA (US); I Peace, Inc., Palo Alto, CA (US)

(21) Appl. No.: 19/237,804

(22) Filed: Jun. 13, 2025

(30) Foreign Application Priority Data
Jun. 14, 2024 (JP) 2024-096465

(51) Int. Cl. C12N 5/074

(52) U.S. Cl. CPC C12N 5/0696; C12N 2506/11

(57) ABSTRACT

[Problem] To provide cells that exhibit reduced immune rejection upon transplantation.

[Solution] A cell that is homozygous for at least one, two, three, four, or all of the group consisting of HLA-A*01:01g, HLA-B*08:01g, HLA-DRB1*03:01, HLA-C*07:01g, HLA-DQB1*02:01g, and HLA-DRB3*01:01.

### TECHNICAL FIELD

The present invention relates to a cell.

### BACKGROUND ART

The major histocompatibility complex (MHC) is known in humans as the human leukocyte antigen (HLA), which is expressed in most cells and tissues. HLA includes six major loci: A, B, C, DR, DQ, and DP. Furthermore, each of these loci comprises numerous different types (alleles), forming complex combinations. Accordingly, there exist tens of thousands of such combinations. HLA plays an essential role in the human immune system, with its principal function being the presentation of antigens for the recognition of self and non-self. When cells or tissues from a donor are transplanted into a recipient, the HLA antigens of the allogeneic cells may be recognized as foreign by immune cells such as T cells, potentially leading to rejection (see, for example, Patent Documents 1 to 5).

### PRIOR ART DOCUMENTS

### Patent Documents

Patent Document 1: Japanese Patent No. 6815453
Patent Document 2: Japanese Patent No. 6933898
Patent Document 3: Japanese Patent No. 7212615
Patent Document 4: Japanese Patent No. 7385281
Patent Document 5: Japanese Patent No. 7440027

### SUMMARY OF THE INVENTION

### Problems to be Solved by the Invention

One object of the present invention is to provide cells that exhibit reduced immune rejection upon transplantation.

### Means for Solving the Problems

According to one aspect of the present invention, there are provided cells that are homozygous for at least one, two, three, four, or all of the alleles selected from the group consisting of HLA-A*01:01g, HLA-B*08:01g, HLA-DRB1*03:01, HLA-C*07:01g, HLA-DQB1*02:01g, and HLA-DRB3*01:01.

The aforementioned cell may be a pluripotent stem cell.

According to one aspect of the present invention, there is provided a cell that is homozygous for HLA-A*01:01g.

The aforementioned cell may further be homozygous for HLA-B*08:01g.

The aforementioned cell may further be homozygous for HLA-DRB1*03:01.

The aforementioned cell may further be homozygous for HLA-C*07:01g.

The aforementioned cell may further be homozygous for HLA-DQB1*02:01g.

The aforementioned cell may further be homozygous for HLA-DRB3*01:01.

The aforementioned cell may be a stem cell.

The aforementioned cell may be a pluripotent stem cell.

According to one aspect of the present invention, there is provided a cell that is homozygous for HLA-B*08:01g.

The aforementioned cell may further be homozygous for HLA-DRB1*03:01.

The aforementioned cell may further be homozygous for HLA-C*07:01g.

The aforementioned cell may further be homozygous for HLA-DQB1*02:01g.

The aforementioned cell may further be homozygous for HLA-DRB3*01:01.

The aforementioned cell may be a stem cell.

The aforementioned cell may be a pluripotent stem cell.

According to one aspect of the present invention, there is provided a cell that is homozygous for HLA-DRB1*03:01.

The aforementioned cell may further be homozygous for HLA-C*07:01g.

The aforementioned cell may further be homozygous for HLA-DQB1*02:01g.

The aforementioned cell may further be homozygous for HLA-DRB3*01:01.

The aforementioned cell may be a stem cell.

The aforementioned cell may be a pluripotent stem cell.

According to one aspect of the present invention, there is provided a cell that is homozygous for HLA-C*07:01g.

The aforementioned cell may further be homozygous for HLA-DQB1*02:01g.

The aforementioned cell may further be homozygous for HLA-DRB3*01:01.

The aforementioned cell may be a stem cell.

The aforementioned cell may be a pluripotent stem cell.

According to one aspect of the present invention, there is provided a cell that is homozygous for HLA-DQB1*02:01g.

The aforementioned cell may further be homozygous for HLA-DRB3*01:01.

The aforementioned cell may be a stem cell.

The aforementioned cell may be a pluripotent stem cell.

According to one aspect of the present invention, there is provided a cell that is homozygous for HLA-DRB3*01:01.

The aforementioned cell may be a stem cell.

The aforementioned cell may be a pluripotent stem cell.

According to one aspect of the present invention, there are provided cells that are homozygous for at least one, two, three, four, or all of the alleles selected from the group consisting of HLA-A*03:01g, HLA-B*07:02g, HLA-DRB1*15:01, HLA-C*07:02g, HLA-DQB1*06:02, and HLA-DRB5*01:01.

The aforementioned cell may be a stem cell.

The aforementioned cell may be a pluripotent stem cell.

According to one aspect of the present invention, there is provided a cell that is homozygous for HLA-A*03:01g.

The aforementioned cell may further be homozygous for HLA-B*07:02g.

The aforementioned cell may further be homozygous for HLA-DRB1*15:01.

The aforementioned cell may further be homozygous for HLA-C*07:02g.

The aforementioned cell may further be homozygous for HLA-DQB1*06:02.

The aforementioned cell may further be homozygous for HLA-DRB5*01:01.

The aforementioned cell may be a stem cell.

The aforementioned cell may be a pluripotent stem cell.

According to one aspect of the present invention, there is provided a cell that is homozygous for HLA-B*07:02g.

The aforementioned cell may further be homozygous for HLA-DRB1*15:01.

The aforementioned cell may further be homozygous for HLA-C*07:02g.

The aforementioned cell may further be homozygous for HLA-DQB1*06:02.

The aforementioned cell may further be homozygous for HLA-DRB5*01:01.

According to one aspect of the present invention, there is provided a cell that is homozygous for HLA-DRB1*15:01.

The aforementioned cell may further be homozygous for HLA-C*07:02g.

The aforementioned cell may further be homozygous for HLA-DQB1*06:02.

The aforementioned cell may further be homozygous for HLA-DRB5*01:01.

The aforementioned cell may be a stem cell.

The aforementioned cell may be a pluripotent stem cell.

According to one aspect of the present invention, there is provided a cell that is homozygous for HLA-C*07:02g.

The aforementioned cell may further be homozygous for HLA-DQB1*06:02.

The aforementioned cell may further be homozygous for HLA-DRB5*01:01.

The aforementioned cell may be a stem cell.

The aforementioned cell may be a pluripotent stem cell.

According to one aspect of the present invention, there is provided a cell that is homozygous for HLA-DQB1*06:02.

The aforementioned cell may further be homozygous for HLA-DRB5*01:01.

The aforementioned cell may be a stem cell.

The aforementioned cell may be a pluripotent stem cell.

According to one aspect of the present invention, there is provided a cell that is homozygous for HLA-DRB5*01:01.

The aforementioned cell may be a pluripotent stem cell.

According to one aspect of the present invention, there are provided cells that are homozygous for at least one, two, three, four, or all of the alleles selected from the group consisting of HLA-A*02:01g, HLA-B*07:02g, HLA-DRB1*15:01, HLA-C*07:02g, HLA-DQB1*06:02, and HLA-DRB5*01:01.

The aforementioned cell may be a stem cell.

The aforementioned cell may be a pluripotent stem cell.

According to one aspect of the present invention, there is provided a cell that is homozygous for HLA-A*02:01g.

The aforementioned cell may further be homozygous for HLA-B*07:02g.

The aforementioned cell may further be homozygous for HLA-DRB1*15:01.

The aforementioned cell may further be homozygous for HLA-C*07:02g.

The aforementioned cell may further be homozygous for HLA-DQB1*06:02.

The aforementioned cell may further be homozygous for HLA-DRB5*01:01.

The aforementioned cell may be a stem cell.

The aforementioned cell may be a pluripotent stem cell.

According to one aspect of the present invention, there is provided a cell that is homozygous for HLA-B*07:02g.

The aforementioned cell may further be homozygous for HLA-DRB1*15:01.

The aforementioned cell may further be homozygous for HLA-C*07:02g.

The aforementioned cell may further be homozygous for HLA-DQB1*06:02.

The aforementioned cell may further be homozygous for HLA-DRB5*01:01.

The aforementioned cell may be a stem cell.

The aforementioned cell may be a pluripotent stem cell.

According to one aspect of the present invention, there is provided a cell that is homozygous for HLA-DRB1*15:01.

The aforementioned cell may further be homozygous for HLA-C*07:02g.

The aforementioned cell may further be homozygous for HLA-DQB1*06:02.

The aforementioned cell may further be homozygous for HLA-DRB5*01:01.

The aforementioned cell may be a stem cell.

The aforementioned cell may be a pluripotent stem cell.

According to one aspect of the present invention, there is provided a cell that is homozygous for HLA-C*07:02g.

The aforementioned cell may further be homozygous for HLA-DQB1*06:02.

The aforementioned cell may further be homozygous for HLA-DRB5*01:01.

The aforementioned cell may be a stem cell.

The aforementioned cell may be a pluripotent stem cell.

According to one aspect of the present invention, there is provided a cell that is homozygous for HLA-DQB1*06:02.

The aforementioned cell may further be homozygous for HLA-DRB5*01:01.

According to one aspect of the present invention, there is provided a cell that is homozygous for HLA-DRB5*01:01.

The aforementioned cell may be a stem cell.

The aforementioned cell may be a pluripotent stem cell.

According to one aspect of the present invention, there are provided cells that are homozygous for at least one, two, three, four, or all of the alleles selected from the group consisting of HLA-A*02:01g, HLA-B*44:02g, HLA-DRB1*04:01, HLA-C*05:01g, HLA-DQB1*03:01g, and HLA-DRB4*01:01g.

The aforementioned cell may be a stem cell.

The aforementioned cell may be a pluripotent stem cell.

According to one aspect of the present invention, there is provided a cell that is homozygous for HLA-A*02:01g.

The aforementioned cell may further be homozygous for HLA-B*44:02g.

The aforementioned cell may further be homozygous for HLA-DRB1*04:01.

The aforementioned cell may further be homozygous for HLA-C*05:01g.

The aforementioned cell may further be homozygous for HLA-DQB1*03:01g.

The aforementioned cell may further be homozygous for HLA-DRB4*01:01g.

The aforementioned cell may be a stem cell.

The aforementioned cell may be a pluripotent stem cell.

According to one aspect of the present invention, there is provided a cell that is homozygous for HLA-B*44:02g.

The aforementioned cell may further be homozygous for HLA-DRB1*04:01.

The aforementioned cell may further be homozygous for HLA-C*05:01g.

The aforementioned cell may further be homozygous for HLA-DQB1*03:01g.

The aforementioned cell may further be homozygous for HLA-DRB4*01:01g.

The aforementioned cell may be a stem cell.

The aforementioned cell may be a pluripotent stem cell.

According to one aspect of the present invention, there is provided a cell that is homozygous for HLA-DRB1*04:01.

The aforementioned cell may further be homozygous for HLA-C*05:01g.

The aforementioned cell may further be homozygous for HLA-DQB1*03:01g.

The aforementioned cell may further be homozygous for HLA-DRB4*01:01g.

The aforementioned cell may be a stem cell.

The aforementioned cell may be a pluripotent stem cell.

According to one aspect of the present invention, there is provided a cell that is homozygous for HLA-C*05:01g.

The aforementioned cell may further be homozygous for HLA-DQB1*03:01g.

The aforementioned cell may further be homozygous for HLA-DRB4*01:01g.

The aforementioned cell may be a stem cell.

The aforementioned cell may be a pluripotent stem cell.

According to one aspect of the present invention, there is provided a cell that is homozygous for HLA-DQB1*03:01g.

The aforementioned cell may further be homozygous for HLA-DRB4*01:01g.

The aforementioned cell may be a stem cell.

The aforementioned cell may be a pluripotent stem cell.

According to one aspect of the present invention, there is provided a cell that is homozygous for HLA-DRB4*01:01g.

The aforementioned cell may be a stem cell.

The aforementioned cell may be a pluripotent stem cell.

According to one aspect of the present invention, there are provided cells that are homozygous for at least one, two, three, four, or all of the alleles selected from the group consisting of HLA-A*29:02g, HLA-B*44:03, HLA-DRB1*07:01, HLA-C*16:01, HLA-DQB1*02:01g, and HLA-DRB4*01:01g.

The aforementioned cell may be a stem cell.

The aforementioned cell may be a pluripotent stem cell.

According to one aspect of the present invention, there is provided a cell that is homozygous for HLA-A*29:02g.

The aforementioned cell may further be homozygous for HLA-B*44:03.

The aforementioned cell may further be homozygous for HLA-DRB1*07:01.

The aforementioned cell may further be homozygous for HLA-C*16:01.

The aforementioned cell may further be homozygous for HLA-DQB1*02:01g.

The aforementioned cell may further be homozygous for HLA-DRB4*01:01g.

According to one aspect of the present invention, there is provided a cell that is homozygous for HLA-B*44:03.

The aforementioned cell may further be homozygous for HLA-DRB1*07:01.

The aforementioned cell may further be homozygous for HLA-C*16:01.

The aforementioned cell may further be homozygous for HLA-DQB1*02:01g.

The aforementioned cell may further be homozygous for HLA-DRB4*01:01g.

The aforementioned cell may be a stem cell.

The aforementioned cell may be a pluripotent stem cell.

According to one aspect of the present invention, there is provided a cell that is homozygous for HLA-DRB1*07:01.

The aforementioned cell may further be homozygous for HLA-C*16:01.

The aforementioned cell may further be homozygous for HLA-DQB1*02:01g.

The aforementioned cell may further be homozygous for HLA-DRB4*01:01g.

The aforementioned cell may be a stem cell.

The aforementioned cell may be a pluripotent stem cell.

According to one aspect of the present invention, there is provided a cell that is homozygous for HLA-C*16:01.

The aforementioned cell may further be homozygous for HLA-DQB1*02:01g.

The aforementioned cell may further be homozygous for HLA-DRB4*01:01g.

The aforementioned cell may be a pluripotent stem cell.

According to one aspect of the present invention, there is provided a cell that is homozygous for HLA-DQB1*02:01g.

The aforementioned cell may further be homozygous for HLA-DRB4*01:01g.

The aforementioned cell may be a stem cell.

The aforementioned cell may be a pluripotent stem cell.

According to one aspect of the present invention, there is provided a cell that is homozygous for HLA-DRB4*01:01g.

The aforementioned cell may be a stem cell.

The aforementioned cell may be a pluripotent stem cell.

According to one aspect of the present invention, there are provided cells that are homozygous for at least one, two, three, four, or all of the alleles selected from the group consisting of HLA-A*03:01g, HLA-B*35:01g, HLA-C*04:01g, HLA-DRB1*01:01, HLA-DQB1*05:01, and HLA-DRBX*NNNN.

The aforementioned cell may be a stem cell.

The aforementioned cell may be a pluripotent stem cell.

According to one aspect of the present invention, there is provided a cell that is homozygous for HLA-A*03:01g.

The aforementioned cell may further be homozygous for HLA-B*35:01g.

The aforementioned cell may further be homozygous for HLA-C*04:01g.

The aforementioned cell may further be homozygous for HLA-DRB1*01:01.

The aforementioned cell may further be homozygous for HLA-DQB1*05:01.

The aforementioned cell may further be homozygous for HLA-DRBX*NNNN.

The aforementioned cell may be a stem cell.

The aforementioned cell may be a pluripotent stem cell.

According to one aspect of the present invention, there is provided a cell that is homozygous for HLA-B*35:01g.

The aforementioned cell may further be homozygous for HLA-C*04:01g.

The aforementioned cell may further be homozygous for HLA-DRB1*01:01.

The aforementioned cell may further be homozygous for HLA-DQB1*05:01.

The aforementioned cell may further be homozygous for HLA-DRBX*NNNN.

The aforementioned cell may be a stem cell.

The aforementioned cell may be a pluripotent stem cell.

According to one aspect of the present invention, there is provided a cell that is homozygous for HLA-C*04:01g.

The aforementioned cell may further be homozygous for HLA-DRB1*01:01.

The aforementioned cell may further be homozygous for HLA-DQB1*05:01.

The aforementioned cell may further be homozygous for HLA-DRBX*NNNN.

The aforementioned cell may be a stem cell.

The aforementioned cell may be a pluripotent stem cell.

According to one aspect of the present invention, there is provided a cell that is homozygous for HLA-DRB1*01:01.

The aforementioned cell may further be homozygous for HLA-DQB1*05:01.

The aforementioned cell may further be homozygous for HLA-DRBX*NNNN.

The aforementioned cell may be a stem cell.

The aforementioned cell may be a pluripotent stem cell.

According to one aspect of the present invention, there is provided a cell that is homozygous for HLA-DQB1*05:01.

The aforementioned cell may further be homozygous for HLA-DRBX*NNNN.

The aforementioned cell may be a stem cell.

The aforementioned cell may be a pluripotent stem cell.

According to one aspect of the present invention, there is provided a cell that is homozygous for HLA-DRBX*NNNN.

The aforementioned cell may be a stem cell.

The aforementioned cell may be a pluripotent stem cell.

According to one aspect of the present invention, there are provided cells that are homozygous for at least one, two, three, four, or all of the alleles selected from the group consisting of HLA-A*02:01g, HLA-B*08:01g, HLA-C*07:01g, HLA-DRB1*03:01, HLA-DQB1*02:01g, and HLA-DRB3*01:01.

The aforementioned cell may be a stem cell.

The aforementioned cell may be a pluripotent stem cell.

According to one aspect of the present invention, there is provided a cell that is homozygous for HLA-A*02:01g.

The aforementioned cell may further be homozygous for HLA-B*08:01g.

The aforementioned cell may further be homozygous for HLA-C*07:01g.

The aforementioned cell may further be homozygous for HLA-DRB1*03:01.

The aforementioned cell may further be homozygous for HLA-DQB1*02:01g.

The aforementioned cell may further be homozygous for HLA-DRB3*01:01.

The aforementioned cell may be a stem cell.

The aforementioned cell may be a pluripotent stem cell.

According to one aspect of the present invention, there is provided a cell that is homozygous for HLA-B*08:01g.

The aforementioned cell may further be homozygous for HLA-C*07:01g.

The aforementioned cell may further be homozygous for HLA-DRB1*03:01.

The aforementioned cell may further be homozygous for HLA-DQB1*02:01g.

The aforementioned cell may further be homozygous for HLA-DRB3*01:01.

The aforementioned cell may be a stem cell.

The aforementioned cell may be a pluripotent stem cell.

According to one aspect of the present invention, there is provided a cell that is homozygous for HLA-C*07:01g.

The aforementioned cell may further be homozygous for HLA-DRB1*03:01.

The aforementioned cell may further be homozygous for HLA-DQB1*02:01g.

The aforementioned cell may further be homozygous for HLA-DRB3*01:01.

According to one aspect of the present invention, there is provided a cell that is homozygous for HLA-DRB1*03:01.

The aforementioned cell may further be homozygous for HLA-DQB1*02:01g.

The aforementioned cell may further be homozygous for HLA-DRB3*01:01.

The aforementioned cell may be a stem cell.

The aforementioned cell may be a pluripotent stem cell.

According to one aspect of the present invention, there is provided a cell that is homozygous for HLA-DQB1*02:01g.

The aforementioned cell may further be homozygous for HLA-DRB3*01:01.

The aforementioned cell may be a stem cell.

The aforementioned cell may be a pluripotent stem cell.

According to one aspect of the present invention, there is provided a cell that is homozygous for HLA-DRB3*01:01.

The aforementioned cell may be a stem cell.

The aforementioned cell may be a pluripotent stem cell.

According to one aspect of the present invention, there are provided cells that are homozygous for at least one, two, three, four, or all of the alleles selected from the group consisting of HLA-A*01:01g, HLA-B*57:01g, HLA-C*06:02g, HLA-DRB1*07:01, HLA-DQB1*03:03g, and HLA-DRB4*01:01g.

The aforementioned cell may be a stem cell.

The aforementioned cell may be a pluripotent stem cell.

According to one aspect of the present invention, there is provided a cell that is homozygous for HLA-A*01:01g.

The aforementioned cell may further be homozygous for HLA-B*57:01g.

The aforementioned cell may further be homozygous for HLA-C*06:02g.

The aforementioned cell may further be homozygous for HLA-DRB1*07:01.

The aforementioned cell may further be homozygous for HLA-DQB1*03:03g.

The aforementioned cell may further be homozygous for HLA-DRB4*01:01g.

The aforementioned cell may be a stem cell.

The aforementioned cell may be a pluripotent stem cell.

According to one aspect of the present invention, there is provided a cell that is homozygous for HLA-B*57:01g.

The aforementioned cell may further be homozygous for HLA-C*06:02g.

The aforementioned cell may further be homozygous for HLA-DRB1*07:01.

The aforementioned cell may further be homozygous for HLA-DQB1*03:03g.

The aforementioned cell may further be homozygous for HLA-DRB4*01:01g.

The aforementioned cell may be a stem cell.

The aforementioned cell may be a pluripotent stem cell.

According to one aspect of the present invention, there is provided a cell that is homozygous for HLA-C*06:02g.

The aforementioned cell may further be homozygous for HLA-DRB1*07:01.

The aforementioned cell may further be homozygous for HLA-DQB1*03:03g.

The aforementioned cell may further be homozygous for HLA-DRB4*01:01g.

The aforementioned cell may be a stem cell.

The aforementioned cell may be a pluripotent stem cell.

According to one aspect of the present invention, there is provided a cell that is homozygous for HLA-DRB1*07:01.

The aforementioned cell may further be homozygous for HLA-DQB1*03:03g.

The aforementioned cell may further be homozygous for HLA-DRB4*01:01g.

The aforementioned cell may be a stem cell.

The aforementioned cell may be a pluripotent stem cell.

According to one aspect of the present invention, there is provided a cell that is homozygous for HLA-DQB1*03:03g.

The aforementioned cell may further be homozygous for HLA-DRB4*01:01g.

The aforementioned cell may be a stem cell.

The aforementioned cell may be a pluripotent stem cell.

According to one aspect of the present invention, there is provided a cell that is homozygous for HLA-DRB4*01:01g.

The aforementioned cell may be a stem cell.

The aforementioned cell may be a pluripotent stem cell.

According to one aspect of the present invention, there are provided cells that are homozygous for at least one, two, three, four, or all of the alleles selected from the group consisting of HLA-A*02:01g, HLA-B*40:01g, HLA-C*03:04g, HLA-DRB1*13:02, HLA-DQB1*06:04g, and HLA-DRB3*03:01.

The aforementioned cell may be a stem cell.

The aforementioned cell may be a pluripotent stem cell.

According to one aspect of the present invention, there is provided a cell that is homozygous for HLA-A*02:01g.

The aforementioned cell may further be homozygous for HLA-B*40:01g.

The aforementioned cell may further be homozygous for HLA-C*03:04g.

The aforementioned cell may further be homozygous for HLA-DRB1*13:02.

The aforementioned cell may further be homozygous for HLA-DQB1*06:04g.

The aforementioned cell may further be homozygous for HLA-DRB3*03:01.

The aforementioned cell may be a stem cell.

The aforementioned cell may be a pluripotent stem cell.

According to one aspect of the present invention, there is provided a cell that is homozygous for HLA-B*40:01g.

The aforementioned cell may further be homozygous for HLA-C*03:04g.

The aforementioned cell may further be homozygous for HLA-DRB1*13:02.

The aforementioned cell may further be homozygous for HLA-DQB1*06:04g.

The aforementioned cell may further be homozygous for HLA-DRB3*03:01.

The aforementioned cell may be a stem cell.

The aforementioned cell may be a pluripotent stem cell.

According to one aspect of the present invention, there is provided a cell that is homozygous for HLA-C*03:04g.

The aforementioned cell may further be homozygous for HLA-DRB1*13:02.

The aforementioned cell may further be homozygous for HLA-DQB1*06:04g.

The aforementioned cell may further be homozygous for HLA-DRB3*03:01.

The aforementioned cell may be a stem cell.

The aforementioned cell may be a pluripotent stem cell.

According to one aspect of the present invention, there is provided a cell that is homozygous for HLA-DRB1*13:02.

The aforementioned cell may further be homozygous for HLA-DQB1*06:04g.

The aforementioned cell may further be homozygous for HLA-DRB3*03:01.

The aforementioned cell may be a stem cell.

The aforementioned cell may be a pluripotent stem cell.

According to one aspect of the present invention, there is provided a cell that is homozygous for HLA-DQB1*06:04g.

The aforementioned cell may further be homozygous for HLA-DRB3*03:01.

The aforementioned cell may be a stem cell.

The aforementioned cell may be a pluripotent stem cell.

According to one aspect of the present invention, there is provided a cell that is homozygous for HLA-DRB3*03:01.

According to one aspect of the present invention, there are provided cells that are homozygous for at least one, two, three, four, or all of the alleles selected from the group consisting of HLA-A*24:02g, HLA-B*07:02g, HLA-C*07:02g, HLA-DRB1*15:01, HLA-DQB1*06:02, and HLA-DRB5*01:01.

The aforementioned cell may be a stem cell.

The aforementioned cell may be a pluripotent stem cell.

According to one aspect of the present invention, there is provided a cell that is homozygous for HLA-A*24:02g.

The aforementioned cell may further be homozygous for HLA-B*07:02g.

The aforementioned cell may further be homozygous for HLA-C*07:02g.

The aforementioned cell may further be homozygous for HLA-DRB1*15:01.

The aforementioned cell may further be homozygous for HLA-DQB1*06:02.

The aforementioned cell may further be homozygous for HLA-DRB5*01:01.

The aforementioned cell may be a stem cell.

The aforementioned cell may be a pluripotent stem cell.

According to one aspect of the present invention, there is provided a cell that is homozygous for HLA-B*07:02g.

The aforementioned cell may further be homozygous for HLA-C*07:02g.

The aforementioned cell may further be homozygous for HLA-DRB1*15:01.

The aforementioned cell may further be homozygous for HLA-DQB1*06:02.

The aforementioned cell may further be homozygous for HLA-DRB5*01:01.

The aforementioned cell may be a pluripotent stem cell.

According to one aspect of the present invention, there is provided a cell that is homozygous for HLA-C*07:02g.

The aforementioned cell may further be homozygous for HLA-DRB1*15:01.

The aforementioned cell may further be homozygous for HLA-DQB1*06:02.

The aforementioned cell may further be homozygous for HLA-DRB5*01:01.

The aforementioned cell may be a stem cell.

The aforementioned cell may be a pluripotent stem cell.

According to one aspect of the present invention, there is provided a cell that is homozygous for HLA-DRB1*15:01.

The aforementioned cell may further be homozygous for HLA-DQB1*06:02.

The aforementioned cell may further be homozygous for HLA-DRB5*01:01.

The aforementioned cell may be a stem cell.

The aforementioned cell may be a pluripotent stem cell.

According to one aspect of the present invention, there is provided a cell that is homozygous for HLA-DQB1*06:02.

The aforementioned cell may further be homozygous for HLA-DRB5*01:01.

The aforementioned cell may be a stem cell.

The aforementioned cell may be a pluripotent stem cell.

According to one aspect of the present invention, there is provided a cell that is homozygous for HLA-DRB5*01:01.

The aforementioned cell may be a stem cell.

The aforementioned cell may be a pluripotent stem cell.

According to one aspect of the present invention, there are provided cells that are homozygous for at least one, two, three, four, or all of the alleles selected from the group consisting of HLA-A*24:02g, HLA-B*52:01g, HLA-DRB1*15:02, HLA-C*12:02, HLA-DRB5*01:02g, and HLA-DQB1*06:01.

The aforementioned cell may be a stem cell.

The aforementioned cell may be a pluripotent stem cell.

According to one aspect of the present invention, there is provided a cell that is homozygous for HLA-A*24:02g.

The aforementioned cell may further be homozygous for HLA-B*52:01g.

The aforementioned cell may further be homozygous for HLA-DRB1*15:02.

The aforementioned cell may further be homozygous for HLA-C*12:02.

The aforementioned cell may further be homozygous for HLA-DRB5*01:02g.

The aforementioned cell may further be homozygous for HLA-DQB1*06:01.

The aforementioned cell may be a stem cell.

The aforementioned cell may be a pluripotent stem cell.

According to one aspect of the present invention, there is provided a cell that is homozygous for HLA-B*52:01g.

The aforementioned cell may further be homozygous for HLA-DRB1*15:02.

The aforementioned cell may further be homozygous for HLA-C*12:02.

The aforementioned cell may further be homozygous for HLA-DRB5*01:02g.

The aforementioned cell may further be homozygous for HLA-DQB1*06:01.

The aforementioned cell may be a stem cell.

The aforementioned cell may be a pluripotent stem cell.

According to one aspect of the present invention, there is provided a cell that is homozygous for HLA-DRB1*15:02.

The aforementioned cell may further be homozygous for HLA-C*12:02.

The aforementioned cell may further be homozygous for HLA-DRB5*01:02g.

The aforementioned cell may further be homozygous for HLA-DQB1*06:01.

The aforementioned cell may be a stem cell.

The aforementioned cell may be a pluripotent stem cell.

According to one aspect of the present invention, there is provided a cell that is homozygous for HLA-C*12:02.

The aforementioned cell may further be homozygous for HLA-DRB5*01:02g.

The aforementioned cell may further be homozygous for HLA-DQB1*06:01.

The aforementioned cell may be a stem cell.

The aforementioned cell may be a pluripotent stem cell.

According to one aspect of the present invention, there is provided a cell that is homozygous for HLA-DRB5*01:02g.

The aforementioned cell may further be homozygous for HLA-DQB1*06:01.

The aforementioned cell may be a pluripotent stem cell.

According to one aspect of the present invention, there is provided a cell that is homozygous for HLA-DQB1*06:01.

The aforementioned cell may be a stem cell.

The aforementioned cell may be a pluripotent stem cell.

According to one aspect of the present invention, there are provided cells that are homozygous for at least one, two, three, four, or all of the alleles selected from the group consisting of HLA-A*33:03g, HLA-B*44:03, HLA-DRB1*13:02, HLA-C*14:03, HLA-DRB3*03:01, and HLA-DQB1*06:04g.

The aforementioned cell may be a stem cell.

The aforementioned cell may be a pluripotent stem cell.

According to one aspect of the present invention, there is provided a cell that is homozygous for HLA-A*33:03g.

The aforementioned cell may further be homozygous for HLA-B*44:03.

The aforementioned cell may further be homozygous for HLA-DRB1*13:02.

The aforementioned cell may further be homozygous for HLA-C*14:03.

The aforementioned cell may further be homozygous for HLA-DRB3*03:01.

The aforementioned cell may further be homozygous for HLA-DQB1*06:04g.

The aforementioned cell may be a stem cell.

The aforementioned cell may be a pluripotent stem cell.

According to one aspect of the present invention, there is provided a cell that is homozygous for HLA-B*44:03.

The aforementioned cell may further be homozygous for HLA-DRB1*13:02.

The aforementioned cell may further be homozygous for HLA-C*14:03.

The aforementioned cell may further be homozygous for HLA-DRB3*03:01.

The aforementioned cell may further be homozygous for HLA-DQB1*06:04g.

The aforementioned cell may be a stem cell.

The aforementioned cell may be a pluripotent stem cell.

According to one aspect of the present invention, there is provided a cell that is homozygous for HLA-DRB1*13:02.

The aforementioned cell may further be homozygous for HLA-C*14:03.

The aforementioned cell may further be homozygous for HLA-DRB3*03:01.

The aforementioned cell may further be homozygous for HLA-DQB1*06:04g.

The aforementioned cell may be a stem cell.

The aforementioned cell may be a pluripotent stem cell.

According to one aspect of the present invention, there is provided a cell that is homozygous for HLA-C*14:03.

The aforementioned cell may further be homozygous for HLA-DRB3*03:01.

The aforementioned cell may further be homozygous for HLA-DQB1*06:04g.

The aforementioned cell may be a stem cell.

The aforementioned cell may be a pluripotent stem cell.

According to one aspect of the present invention, there is provided a cell that is homozygous for HLA-DRB3*03:01.

The aforementioned cell may further be homozygous for HLA-DQB1*06:04g.

The aforementioned cell may be a stem cell.

The aforementioned cell may be a pluripotent stem cell.

According to one aspect of the present invention, there is provided a cell that is homozygous for HLA-DQB1*06:04g.

The aforementioned cell may be a stem cell.

The aforementioned cell may be a pluripotent stem cell.

According to one aspect of the present invention, there are provided cells that are homozygous for at least one, two, three, four, or all of the alleles selected from the group consisting of HLA-A*24:02g, HLA-B*07:02g, HLA-DRB1*01:01, HLA-C*07:02g, HLA-DRBX*NNNN, and HLA-DQB1*05:01.

The aforementioned cell may be a stem cell.

The aforementioned cell may be a pluripotent stem cell.

According to one aspect of the present invention, there is provided a cell that is homozygous for HLA-A*24:02g.

The aforementioned cell may further be homozygous for HLA-B*07:02g.

The aforementioned cell may further be homozygous for HLA-DRB1*01:01.

The aforementioned cell may further be homozygous for HLA-C*07:02g.

The aforementioned cell may further be homozygous for HLA-DRBX*NNNN.

The aforementioned cell may further be homozygous for HLA-DQB1*05:01.

The aforementioned cell may be a pluripotent stem cell.

According to one aspect of the present invention, there is provided a cell that is homozygous for HLA-B*07:02g.

The aforementioned cell may further be homozygous for HLA-DRB1*01:01.

The aforementioned cell may further be homozygous for HLA-C*07:02g.

The aforementioned cell may further be homozygous for HLA-DRBX*NNNN.

The aforementioned cell may further be homozygous for HLA-DQB1*05:01.

The aforementioned cell may be a stem cell.

The aforementioned cell may be a pluripotent stem cell.

According to one aspect of the present invention, there is provided a cell that is homozygous for HLA-DRB1*01:01.

The aforementioned cell may further be homozygous for HLA-C*07:02g.

The aforementioned cell may further be homozygous for HLA-DRBX*NNNN.

The aforementioned cell may further be homozygous for HLA-DQB1*05:01.

The aforementioned cell may be a stem cell.

The aforementioned cell may be a pluripotent stem cell.

According to one aspect of the present invention, there is provided a cell that is homozygous for HLA-C*07:02g.

The aforementioned cell may further be homozygous for HLA-DRBX*NNNN.

The aforementioned cell may further be homozygous for HLA-DQB1*05:01.

The aforementioned cell may be a stem cell.

The aforementioned cell may be a pluripotent stem cell.

According to one aspect of the present invention, there is provided a cell that is homozygous for HLA-DRBX*NNNN.

The aforementioned cell may further be homozygous for HLA-DQB1*05:01.

The aforementioned cell may be a stem cell.

The aforementioned cell may be a pluripotent stem cell.

According to one aspect of the present invention, there is provided a cell that is homozygous for HLA-DQB1*05:01.

The aforementioned cell may be a stem cell.

The aforementioned cell may be a pluripotent stem cell.

According to one aspect of the present invention, there are provided cells that are homozygous for at least one, two, three, four, or all of the alleles selected from the group consisting of HLA-A*24:02g, HLA-B*54:01g, HLA-DRB1*04:05, HLA-C*01:02g, HLA-DRB4*01:01g, and HLA-DQB1*04:01g.

The aforementioned cell may be a stem cell.

The aforementioned cell may be a pluripotent stem cell.

According to one aspect of the present invention, there is provided a cell that is homozygous for HLA-A*24:02g.

The aforementioned cell may further be homozygous for HLA-B*54:01g.

The aforementioned cell may further be homozygous for HLA-DRB1*04:05.

The aforementioned cell may further be homozygous for HLA-C*01:02g.

The aforementioned cell may further be homozygous for HLA-DRB4*01:01g.

The aforementioned cell may further be homozygous for HLA-DQB1*04:01g.

The aforementioned cell may be a stem cell.

The aforementioned cell may be a pluripotent stem cell.

According to one aspect of the present invention, there is provided a cell that is homozygous for HLA-B*54:01g.

The aforementioned cell may further be homozygous for HLA-DRB1*04:05.

The aforementioned cell may further be homozygous for HLA-C*01:02g.

The aforementioned cell may further be homozygous for HLA-DRB4*01:01g.

The aforementioned cell may further be homozygous for HLA-DQB1*04:01g.

The aforementioned cell may be a stem cell.

The aforementioned cell may be a pluripotent stem cell.

According to one aspect of the present invention, there is provided a cell that is homozygous for HLA-DRB1*04:05.

The aforementioned cell may further be homozygous for HLA-C*01:02g.

The aforementioned cell may further be homozygous for HLA-DRB4*01:01g.

The aforementioned cell may further be homozygous for HLA-DQB1*04:01g.

The aforementioned cell may be a stem cell.

The aforementioned cell may be a pluripotent stem cell.

According to one aspect of the present invention, there is provided a cell that is homozygous for HLA-C*01:02g.

The aforementioned cell may further be homozygous for HLA-DRB4*01:01g.

The aforementioned cell may further be homozygous for HLA-DQB1*04:01g.

The aforementioned cell may be a stem cell.

The aforementioned cell may be a pluripotent stem cell.

According to one aspect of the present invention, there is provided a cell that is homozygous for HLA-DRB4*01:01g.

The aforementioned cell may further be homozygous for HLA-DQB1*04:01g.

The aforementioned cell may be a stem cell.

The aforementioned cell may be a pluripotent stem cell.

According to one aspect of the present invention, there is provided a cell that is homozygous for HLA-DQB1*04:01g.

The aforementioned cell may be a stem cell.

The aforementioned cell may be a pluripotent stem cell.

According to one aspect of the present invention, there are provided cells that are homozygous for at least one, two, three, four, or all of the alleles selected from the group consisting of HLA-A*24:02g, HLA-B*59:01, HLA-DRB1*04:05, HLA-C*01:02g, HLA-DRB4*01:01g, and HLA-DQB1*04:01g.

The aforementioned cell may be a stem cell.

The aforementioned cell may be a pluripotent stem cell.

According to one aspect of the present invention, there is provided a cell that is homozygous for HLA-A*24:02g.

The aforementioned cell may further be homozygous for HLA-B*59:01.

The aforementioned cell may further be homozygous for HLA-DRB1*04:05.

The aforementioned cell may further be homozygous for HLA-C*01:02g.

The aforementioned cell may further be homozygous for HLA-DRB4*01:01g.

The aforementioned cell may further be homozygous for HLA-DQB1*04:01g.

The aforementioned cell may be a stem cell.

The aforementioned cell may be a pluripotent stem cell.

According to one aspect of the present invention, there is provided a cell that is homozygous for HLA-B*59:01.

The aforementioned cell may further be homozygous for HLA-DRB1*04:05.

The aforementioned cell may further be homozygous for HLA-C*01:02g.

The aforementioned cell may further be homozygous for HLA-DRB4*01:01g.

The aforementioned cell may further be homozygous for HLA-DQB1*04:01g.

The aforementioned cell may be a stem cell.

The aforementioned cell may be a pluripotent stem cell.

According to one aspect of the present invention, there is provided a cell that is homozygous for HLA-DRB1*04:05.

The aforementioned cell may further be homozygous for HLA-C*01:02g.

The aforementioned cell may further be homozygous for HLA-DRB4*01:01g.

The aforementioned cell may further be homozygous for HLA-DQB1*04:01g.

The aforementioned cell may be a pluripotent stem cell.

According to one aspect of the present invention, there is provided a cell that is homozygous for HLA-C*01:02g.

The aforementioned cell may further be homozygous for HLA-DRB4*01:01g.

The aforementioned cell may further be homozygous for HLA-DQB1*04:01g.

The aforementioned cell may be a stem cell.

The aforementioned cell may be a pluripotent stem cell.

According to one aspect of the present invention, there is provided a cell that is homozygous for HLA-DRB4*01:01g.

The aforementioned cell may further be homozygous for HLA-DQB1*04:01g.

The aforementioned cell may be a stem cell.

The aforementioned cell may be a pluripotent stem cell.

According to one aspect of the present invention, there is provided a cell that is homozygous for HLA-DQB1*04:01g.

The aforementioned cell may be a stem cell.

The aforementioned cell may be a pluripotent stem cell.

According to one aspect of the present invention, there are provided cells that are homozygous for at least one, two, three, four, or all of the alleles selected from the group consisting of HLA-A*02:07g, HLA-C*01:02g, HLA-B*46:01g, HLA-DRBX*NNNN, HLA-DRB1*08:03, and HLA-DQB1*06:01.

According to one aspect of the present invention, there is provided a cell that is homozygous for HLA-A*02:07g.

The aforementioned cell may further be homozygous for HLA-C*01:02g.

The aforementioned cell may further be homozygous for HLA-B*46:01g.

The aforementioned cell may further be homozygous for HLA-DRBX*NNNN.

The aforementioned cell may further be homozygous for HLA-DRB1*08:03.

The aforementioned cell may further be homozygous for HLA-DQB1*06:01.

The aforementioned cell may be a stem cell.

The aforementioned cell may be a pluripotent stem cell.

According to one aspect of the present invention, there is provided a cell that is homozygous for HLA-C*01:02g.

The aforementioned cell may further be homozygous for HLA-B*46:01g.

The aforementioned cell may further be homozygous for HLA-DRBX*NNNN.

The aforementioned cell may further be homozygous for HLA-DRB1*08:03.

The aforementioned cell may further be homozygous for HLA-DQB1*06:01.

The aforementioned cell may be a stem cell.

The aforementioned cell may be a pluripotent stem cell.

According to one aspect of the present invention, there is provided a cell that is homozygous for HLA-B*46:01g.

The aforementioned cell may further be homozygous for HLA-DRBX*NNNN.

The aforementioned cell may further be homozygous for HLA-DRB1*08:03.

The aforementioned cell may further be homozygous for HLA-DQB1*06:01.

The aforementioned cell may be a stem cell.

The aforementioned cell may be a pluripotent stem cell.

According to one aspect of the present invention, there is provided a cell that is homozygous for HLA-DRBX*NNNN.

The aforementioned cell may further be homozygous for HLA-DRB1*08:03.

The aforementioned cell may further be homozygous for HLA-DQB1*06:01.

The aforementioned cell may be a stem cell.

The aforementioned cell may be a pluripotent stem cell.

According to one aspect of the present invention, there is provided a cell that is homozygous for HLA-DRB1*08:03.

The aforementioned cell may further be homozygous for HLA-DQB1*06:01.

The aforementioned cell may be a stem cell.

The aforementioned cell may be a pluripotent stem cell.

According to one aspect of the present invention, there is provided a cell that is homozygous for HLA-DQB1*06:01.

The aforementioned cell may be a stem cell.

The aforementioned cell may be a pluripotent stem cell.

According to one aspect of the present invention, there are provided cells that are homozygous for at least one, two, three, four, or all of the alleles selected from the group consisting of HLA-A*11:01g, HLA-C*04:01g, HLA-B*15:01g, HLA-DRB4*01:01g, HLA-DRB1*04:06, and HLA-DQB103:02g.

The aforementioned cell may be a stem cell.

The aforementioned cell may be a pluripotent stem cell.

According to one aspect of the present invention, there is provided a cell that is homozygous for HLA-A*11:01g.

The aforementioned cell may further be homozygous for HLA-C*04:01g.

The aforementioned cell may further be homozygous for HLA-B*15:01g.

The aforementioned cell may further be homozygous for HLA-DRB4*01:01g.

The aforementioned cell may further be homozygous for HLA-DRB1*04:06.

The aforementioned cell may further be homozygous for HLA-DQB1*03:02g.

The aforementioned cell may be a stem cell.

The aforementioned cell may be a pluripotent stem cell.

According to one aspect of the present invention, there is provided a cell that is homozygous for HLA-C*04:01g.

The aforementioned cell may further be homozygous for HLA-B*15:01g.

The aforementioned cell may further be homozygous for HLA-DRB4*01:01g.

The aforementioned cell may further be homozygous for HLA-DRB1*04:06.

The aforementioned cell may further be homozygous for HLA-DQB1*03:02g.

According to one aspect of the present invention, there is provided a cell that is homozygous for HLA-B*15:01g.

The aforementioned cell may further be homozygous for HLA-DRB4*01:01g.

The aforementioned cell may further be homozygous for HLA-DRB1*04:06.

The aforementioned cell may further be homozygous for HLA-DQB1*03:02g.

The aforementioned cell may be a stem cell.

The aforementioned cell may be a pluripotent stem cell.

According to one aspect of the present invention, there is provided a cell that is homozygous for HLA-DRB4*01:01g.

The aforementioned cell may further be homozygous for HLA-DRB1*04:06.

The aforementioned cell may further be homozygous for HLA-DQB1*03:02g.

The aforementioned cell may be a stem cell.

The aforementioned cell may be a pluripotent stem cell.

According to one aspect of the present invention, there is provided a cell that is homozygous for HLA-DRB1*04:06.

The aforementioned cell may further be homozygous for HLA-DQB1*03:02g.

The aforementioned cell may be a stem cell.

The aforementioned cell may be a pluripotent stem cell.

According to one aspect of the present invention, there is provided a cell that is homozygous for HLA-DQB1*03:02g.

The aforementioned cell may be a pluripotent stem cell.

According to one aspect of the present invention, there is provided a cell that is homozygous for at least one, two, three, four, or all of the alleles selected from the group consisting of HLA-A*11:01g, HLA-C*01:02g, HLA-B*54:01g, HLA-DRB4*01:01g, HLA-DRB1*04:05, and HLA-DQB1*04:01g.

The aforementioned cell may be a stem cell.

The aforementioned cell may be a pluripotent stem cell.

According to one aspect of the present invention, there is provided a cell that is homozygous for HLA-A*11:01g.

The aforementioned cell may further be homozygous for HLA-C*01:02g.

The aforementioned cell may further be homozygous for HLA-B*54:01g.

The aforementioned cell may further be homozygous for HLA-DRB4*01:01g.

The aforementioned cell may further be homozygous for HLA-DRB1*04:05.

The aforementioned cell may further be homozygous for HLA-DQB1*04:01g.

The aforementioned cell may be a stem cell.

The aforementioned cell may be a pluripotent stem cell.

According to one aspect of the present invention, there is provided a cell that is homozygous for HLA-C*01:02g.

The aforementioned cell may further be homozygous for HLA-B*54:01g.

The aforementioned cell may further be homozygous for HLA-DRB4*01:01g.

The aforementioned cell may further be homozygous for HLA-DRB1*04:05.

The aforementioned cell may further be homozygous for HLA-DQB1*04:01g.

The aforementioned cell may be a stem cell.

The aforementioned cell may be a pluripotent stem cell.

According to one aspect of the present invention, there is provided a cell that is homozygous for HLA-B*54:01g.

The aforementioned cell may further be homozygous for HLA-DRB4*01:01g.

The aforementioned cell may further be homozygous for HLA-DRB1*04:05.

The aforementioned cell may further be homozygous for HLA-DQB1*04:01g.

The aforementioned cell may be a stem cell.

The aforementioned cell may be a pluripotent stem cell.

According to one aspect of the present invention, there is provided a cell that is homozygous for HLA-DRB4*01:01g.

The aforementioned cell may further be homozygous for HLA-DRB1*04:05.

The aforementioned cell may further be homozygous for HLA-DQB1*04:01g.

The aforementioned cell may be a stem cell.

The aforementioned cell may be a pluripotent stem cell.

According to one aspect of the present invention, there is provided a cell that is homozygous for HLA-DRB1*04:05.

The aforementioned cell may further be homozygous for HLA-DQB1*04:01g.

According to one aspect of the present invention, there is provided a cell that is homozygous for HLA-DQB1*04:01g.

The aforementioned cell may be a stem cell.

The aforementioned cell may be a pluripotent stem cell.

According to one aspect of the present invention, there is provided a cell that is homozygous for at least one, two, three, four, or all of the alleles selected from the group consisting of HLA-A*26:01g, HLA-C*03:04g, HLA-B*40:02g, HLA-DRB4*01:01g, HLA-DRB1*09:01, and HLA-DQB1*03:03g.

The aforementioned cell may be a stem cell.

The aforementioned cell may be a pluripotent stem cell.

According to one aspect of the present invention, there is provided a cell that is homozygous for HLA-A*26:01g.

The aforementioned cell may further be homozygous for HLA-C*03:04g.

The aforementioned cell may further be homozygous for HLA-B*40:02g.

The aforementioned cell may further be homozygous for HLA-DRB4*01:01g.

The aforementioned cell may further be homozygous for HLA-DRB1*09:01.

The aforementioned cell may further be homozygous for HLA-DQB1*03:03g.

The aforementioned cell may be a stem cell.

The aforementioned cell may be a pluripotent stem cell.

According to one aspect of the present invention, there is provided a cell that is homozygous for HLA-C*03:04g.

The aforementioned cell may further be homozygous for HLA-B*40:02g.

The aforementioned cell may further be homozygous for HLA-DRB4*01:01g.

The aforementioned cell may further be homozygous for HLA-DRB1*09:01.

The aforementioned cell may further be homozygous for HLA-DQB1*03:03g.

The aforementioned cell may be a stem cell.

The aforementioned cell may be a pluripotent stem cell.

According to one aspect of the present invention, there is provided a cell that is homozygous for HLA-B*40:02g.

The aforementioned cell may further be homozygous for HLA-DRB4*01:01g.

The aforementioned cell may further be homozygous for HLA-DRB1*09:01.

The aforementioned cell may further be homozygous for HLA-DQB1*03:03g.

The aforementioned cell may be a stem cell.

The aforementioned cell may be a pluripotent stem cell.

According to one aspect of the present invention, there is provided a cell that is homozygous for HLA-DRB4*01:01g.

The aforementioned cell may further be homozygous for HLA-DRB1*09:01.

The aforementioned cell may further be homozygous for HLA-DQB1*03:03g.

The aforementioned cell may be a stem cell.

The aforementioned cell may be a pluripotent stem cell.

According to one aspect of the present invention, there is provided a cell that is homozygous for HLA-DRB1*09:01.

The aforementioned cell may further be homozygous for HLA-DQB1*03:03g.

The aforementioned cell may be a stem cell.

The aforementioned cell may be a pluripotent stem cell.

According to one aspect of the present invention, there is provided a cell that is homozygous for HLA-DQB1*03:03g.

The aforementioned cell may be a stem cell.

The aforementioned cell may be a pluripotent stem cell.

According to one aspect of the present invention, there is provided a cell that is homozygous for at least one, two, three, four, or all of the alleles selected from the group consisting of HLA-A*24:02g, HLA-C*08:01g, HLA-B*40:06, HLA-DRB4*01:01g, HLA-DRB1*09:01, and HLA-DQB1*03:03g.

The aforementioned cell may be a stem cell.

The aforementioned cell may be a pluripotent stem cell.

According to one aspect of the present invention, there is provided a cell that is homozygous for HLA-A*24:02g.

The aforementioned cell may further be homozygous for HLA-C*08:01g.

The aforementioned cell may further be homozygous for HLA-B*40:06.

The aforementioned cell may further be homozygous for HLA-DRB4*01:01g.

The aforementioned cell may further be homozygous for HLA-DRB1*09:01.

The aforementioned cell may further be homozygous for HLA-DQB1*03:03g.

According to one aspect of the present invention, there is provided a cell that is homozygous for HLA-C*08:01g.

The aforementioned cell may further be homozygous for HLA-B*40:06.

The aforementioned cell may further be homozygous for HLA-DRB4*01:01g.

The aforementioned cell may further be homozygous for HLA-DRB1*09:01.

The aforementioned cell may further be homozygous for HLA-DQB1*03:03g.

The aforementioned cell may be a stem cell.

The aforementioned cell may be a pluripotent stem cell.

According to one aspect of the present invention, there is provided a cell that is homozygous for HLA-B*40:06.

The aforementioned cell may further be homozygous for HLA-DRB4*01:01g.

The aforementioned cell may further be homozygous for HLA-DRB1*09:01.

The aforementioned cell may further be homozygous for HLA-DQB1*03:03g.

The aforementioned cell may be a stem cell.

The aforementioned cell may be a pluripotent stem cell.

According to one aspect of the present invention, there is provided a cell that is homozygous for HLA-DRB4*01:01g.

The aforementioned cell may further be homozygous for HLA-DRB1*09:01.

The aforementioned cell may further be homozygous for HLA-DQB1*03:03g.

According to one aspect of the present invention, there is provided a cell that is homozygous for HLA-DRB1*09:01.

The aforementioned cell may further be homozygous for HLA-DQB1*03:03g.

The aforementioned cell may be a stem cell.

The aforementioned cell may be a pluripotent stem cell.

According to one aspect of the present invention, there is provided a cell that is homozygous for HLA-DQB1*03:03g.

The aforementioned cell may be a stem cell.

The aforementioned cell may be a pluripotent stem cell.

According to one aspect of the present invention, there is provided a cell that is homozygous for at least one, two, or all of the group consisting of HLA-A*02:01, HLA-C*06:02, and HLA-DPA1*01:03.

The aforementioned cell may be a stem cell.

The aforementioned cell may be a pluripotent stem cell.

According to one aspect of the present invention, there is provided a cell that is homozygous for HLA-A*02:01.

The aforementioned cell may further be homozygous for HLA-C*06:02.

The aforementioned cell may further be homozygous for HLA-DPA1*01:03.

The aforementioned cell may be a stem cell.

The aforementioned cell may be a pluripotent stem cell.

According to one aspect of the present invention, there is provided a cell that is homozygous for HLA-C*06:02.

The aforementioned cell may further be homozygous for HLA-DPA1*01:03.

The aforementioned cell may be a stem cell.

The aforementioned cell may be a pluripotent stem cell.

According to one aspect of the present invention, there is provided a cell that is homozygous for HLA-DPA1*01:03.

The aforementioned cell may be a stem cell.

The aforementioned cell may be a pluripotent stem cell.

### Effect of the Invention

According to the present invention, it is possible to provide cells that exhibit reduced rejection responses upon transplantation.

### BRIEF DESCRIPTION OF THE DRAWINGS

FIG. 1 is a table showing combinations of HLA haplotype homozygotes in Caucasian donors according to Example 1.

FIG. 2 is a table showing the allele frequencies of each Caucasian donor according to Example 1.

FIG. 3 is a table showing the allele frequencies of each Caucasian donor according to Example 1.

FIG. 4 is a table showing combinations of HLA haplotype homozygotes in Japanese individuals according to Example 3.

FIG. 5 is a table showing the allele frequencies of each Japanese donor according to Example 3.

FIG. 6 is a table showing the allele frequencies of each Caucasian donor according to Example 5.

### MODES FOR CARRYING OUT THE INVENTION

Embodiments of the present invention are described below. However, it should not be construed that the following embodiments limit the present invention. From the present disclosure, those skilled in the art will recognize various alternative embodiments, examples, and operational techniques. It should be understood that the present invention encompasses various embodiments and the like not described herein.

A cell according to a first embodiment of the present invention is homozygous for HLA-A*01:01g, HLA-B*08:01g, and HLA-DRB1*03:01. The cell homozygous for HLA-A*01:01g, HLA-B*08:01g, and HLA-DRB1*03:01 may also be homozygous for HLA-C*07:01g, may also be homozygous for HLA-DQB1*02:01g, and may also be homozygous for HLA-DRB3*01:01.

A cell according to a second embodiment of the present invention is homozygous for HLA-A*03:01g, HLA-B*07:02g, and HLA-DRB1*15:01. The cell homozygous for HLA-A*03:01g, HLA-B*07:02g, and HLA-DRB1*15:01 may also be homozygous for HLA-C*07:02g, may also be homozygous for HLA-DQB1*06:02, and may also be homozygous for HLA-DRB5*01:01.

A cell according to a third embodiment of the present invention is homozygous for HLA-A*02:01g, HLA-B*07:02g, and HLA-DRB1*15:01. The cell homozygous for HLA-A*02:01g, HLA-B*07:02g, and HLA-DRB1*15:01 may also be homozygous for HLA-C*07:02g, may also be homozygous for HLA-DQB1*06:02, and may also be homozygous for HLA-DRB5*01:01.

A cell according to a fourth embodiment of the present invention is homozygous for HLA-A*02:01g, HLA-B*44:02g, and HLA-DRB1*04:01. The cell homozygous for HLA-A*02:01g, HLA-B*44:02g, and HLA-DRB1*04:01 may also be homozygous for HLA-C*05:01g, may also be homozygous for HLA-DQB1*03:01g, and may also be homozygous for HLA-DRB4*01:01g.

A cell according to a fifth embodiment of the present invention is homozygous for HLA-A*29:02g, HLA-B*44:03, and HLA-DRB1*07:01. The cell homozygous for HLA-A*29:02g, HLA-B*44:03, and HLA-DRB1*07:01 may also be homozygous for HLA-C*16:01, may also be homozygous for HLA-DQB1*02:01g, and may also be homozygous for HLA-DRB4*01:01g.

A cell according to a sixth embodiment of the present invention is homozygous for HLA-A*03:01g, HLA-B*35:01g, and HLA-DRB1*01:01. The cell homozygous for HLA-A*03:01g, HLA-B*35:01g, and HLA-DRB1*01:01 may also be homozygous for HLA-C*04:01g, may also be homozygous for HLA-DQB1*05:01, and may also be homozygous for HLA-DRBX*NNNN.

A cell according to a seventh embodiment of the present invention is homozygous for HLA-A*02:01g, HLA-B*08:01g, and HLA-DRB1*03:01. The cell homozygous for HLA-A*02:01g, HLA-B*08:01g, and HLA-DRB1*03:01 may also be homozygous for HLA-C*07:01g, may also be homozygous for HLA-DQB1*02:01g, and may also be homozygous for HLA-DRB3*01:01.

A cell according to an eighth embodiment of the present invention is homozygous for HLA-A*01:01g, HLA-B*57:01g, and HLA-DRB1*07:01. The cell homozygous for HLA-A*01:01g, HLA-B*57:01g, and HLA-DRB1*07:01 may also be homozygous for HLA-C*06:02g, may also be homozygous for HLA-DQB1*03:03g, and may also be homozygous for HLA-DRB4*01:01g.

A cell according to a ninth embodiment of the present invention is homozygous for HLA-A*02:01g, HLA-B*40:01g, and HLA-DRB1*13:02. The cell homozygous for HLA-A*02:01g, HLA-B*40:01g, and HLA-DRB1*13:02 may also be homozygous for HLA-C*03:04g, may also be homozygous for HLA-DQB1*06:04g, and may also be homozygous for HLA-DRB3*03:01.

A cell according to a tenth embodiment of the present invention is homozygous for HLA-A*24:02g, HLA-B*07:02g, and HLA-DRB1*15:01. The cell homozygous for HLA-A*24:02g, HLA-B*07:02g, and HLA-DRB1*15:01 may also be homozygous for HLA-C*07:02g, may also be homozygous for HLA-DQB1*06:02, and may also be homozygous for HLA-DRB5*01:01.

In the Caucasoid population, the frequency of each allele combination of the cells according to the first through tenth embodiments is high. For example, the frequency of each allele combination of the cells according to the first through tenth embodiments in the Caucasoid population is 2.0% or more, 2.5% or more, 3.0% or more, 3.5% or more, 4.0% or more, 4.5% or more, 5.0% or more, 5.5% or more, 6.0% or more, 6.5% or more, 7.0% or more, 7.5% or more, 8.0% or more, 8.5% or more, 9.0% or more, 9.5% or more, 10.0% or more, 11.0% or more, 12.0% or more, 13.0% or more, 14.0% or more, 15.0% or more, 16.0% or more, 17.0% or more, 18.0% or more, 20.0% or more, 22.0% or more, 24.0% or more, or 26.0% or more. Therefore, the cells according to the first through tenth embodiments are suitable for transplantation into individuals of the Caucasoid population. In addition, by combining at least some of the cells according to the first through tenth embodiments, it is possible to cover a large proportion of the Caucasoid population.

A cell according to an eleventh embodiment of the present invention is homozygous for HLA-A*24:02g, HLA-B*52:01g, and HLA-DRB1*15:02. The cell homozygous for HLA-A*24:02g, HLA-B*52:01g, and HLA-DRB1*15:02 may also be homozygous for HLA-C*12:02, may also be homozygous for HLA-DRB5*01:02g, and may also be homozygous for HLA-DQB1*06:01.

A cell according to a twelfth embodiment of the present invention is homozygous for HLA-A*33:03g, HLA-B*44:03, and HLA-DRB1*13:02. The cell homozygous for HLA-A*33:03g, HLA-B*44:03, and HLA-DRB1*13:02 may also be homozygous for HLA-C*14:03, may also be homozygous for HLA-DRB3*03:01, and may also be homozygous for HLA-DQB1*06:04g.

A cell according to a thirteenth embodiment of the present invention is homozygous for HLA-A*24:02g, HLA-B*07:02g, and HLA-DRB1*01:01. The cell homozygous for HLA-A*24:02g, HLA-B*07:02g, and HLA-DRB1*01:01 may also be homozygous for HLA-C*07:02g, may also be homozygous for HLA-DRBX*NNNN, and may also be homozygous for HLA-DQB1*05:01.

The cell according to a fourteenth embodiment of the present invention is homozygous for HLA-A*24:02g, HLA-B*54:01g, and HLA-DRB1*04:05. The cell homozygous for HLA-A*24:02g, HLA-B*54:01g, and HLA-DRB1*04:05 may also be homozygous for HLA-C*01:02g, may also be homozygous for HLA-DRB4*01:01g, and may also be homozygous for HLA-DQB1*04:01g.

A cell according to a fifteenth embodiment of the present invention is homozygous for HLA-A*24:02g, HLA-B*59:01, and HLA-DRB1*04:05. The cell homozygous for HLA-A*24:02g, HLA-B*59:01, and HLA-DRB1*04:05 may also be homozygous for HLA-C*01:02g, may also be homozygous for HLA-DRB4*01:01g, and may also be homozygous for HLA-DQB1*04:01g.

A cell according to a sixteenth embodiment of the present invention is homozygous for HLA-A*02:07g, HLA-B*46:01g, and HLA-DRB1*08:03. The cell homozygous for HLA-A*02:07g, HLA-B*46:01g, and HLA-DRB1*08:03 may also be homozygous for HLA-C*01:02g, may also be homozygous for HLA-DRBX*NNNN, and may also be homozygous for HLA-DQB1*06:01.

In the Japanese population, the frequencies of the allele combinations for each of the cells according to the eleventh to twentieth embodiments are high. For example, in the Japanese population, the frequencies of the allele combinations for each of the cells according to the eleventh to twentieth embodiments are 2.0% or more, 2.5% or more, 3.0% or more, 3.5% or more, 4.0% or more, 4.5% or more, 5.0% or more, 5.5% or more, 6.0% or more, 6.5% or more, 7.0% or more, 7.5% or more, 8.0% or more, 8.5% or more, 9.0% or more, 9.5% or more, 10.0% or more, 11.0% or more, 12.0% or more, 13.0% or more, 14.0% or more, 15.0% or more, 16.0% or more, 17.0% or more, 18.0% or more, 20.0% or more, 22.0% or more, 24.0% or more, or 26.0% or more, or 27.0%. Accordingly, the cells according to the eleventh to twentieth embodiments are suitable for transplantation into Japanese individuals. Moreover, by combining at least some of the cells according to the eleventh to twentieth embodiments, it is possible to cover a large proportion of the Japanese population.

A cell according to a twenty-first embodiment of the present invention is homozygous for HLA-A*02:01, HLA-C*06:02, and HLA-DPA1*01:03.

In the Caucasoid population, the frequency of the allele combination of the cell according to the twenty-first embodiment is high. For example, in the Caucasoid population, the frequency of the allele combination of the cell according to the twenty-first embodiment is 2.0% or more, 2.5% or more, 3.0% or more, 3.5% or more, 4.0% or more, 4.5% or more, 5.0% or more, 5.5% or more, 6.0% or more, 6.5% or more, 7.0% or more, 7.5% or more, 8.0% or more, 8.5% or more, 9.0% or more, 9.5% or more, 10.0% or more, 11.0% or more, 12.0% or more, 13.0% or more, 14.0% or more, 15.0% or more, 16.0% or more, 17.0% or more, 18.0% or more, 20.0% or more, 22.0% or more, 24.0% or more, or 26.0% or more. Accordingly, the cell according to the twenty-first embodiment is suitable for transplantation into Caucasoid individuals.

The cells according to the first to twenty-first embodiments may be of human origin or may be derived from non-human animals. The cells according to the first to twenty-first embodiments may be somatic cells. Examples of somatic cells include, but are not limited to, fibroblasts, blood cells, dental pulp stem cells, keratinocytes, dermal papilla cells, oral epithelial cells, somatic stem/progenitor cells, and urinary cells. The blood cells may be nucleated cells such as monocytes, neutrophils, eosinophils, basophils, and lymphocytes. The blood cells may be, for example, vascular endothelial progenitor cells, hematopoietic stem/progenitor cells, T cells, or B cells. The T cells may be αβ T cells or γδ T cells. The blood cells may also be blood cells excluding T cells.

The cells according to the first to twenty-first embodiments may be stem cells. The stem cells may be pluripotent stem cells. The pluripotent stem cells may, for example, have a positivity rate of 30% or more, 50% or more, preferably 80% or more, for any of TRA1-60, OCT3/4, SSEA3, SSEA4, TRA1-81, LIN28, and NANOG. Examples of pluripotent stem cells include induced pluripotent stem (iPS) cells and embryonic stem (ES) cells. iPS cells are induced by introducing reprogramming factors into somatic cells. In the context of inducing stem cells, the term “induction” refers to reprogramming, initialization, transdifferentiation, and cell fate reprogramming.

The reprogramming factors may include, for example, OCT3/4, SOX2, KLF4, and c-MYC. As a reprogramming factor, a modified version of OCT3/4, referred to as M30, may be used. The reprogramming factors may also include at least one selected from the group consisting of LIN28A, FOXHI, LIN28B, GLIS1, p53-dominant negative, p53-P275S, L-MYC, NANOG, DPPA2, DPPA4, DPPA5, ZIC3, BCL-2, E-RAS, TPT1, SALL2, NACI, DAX1, TERT, ZNF206, FOXD3, REX1, UTF1, KLF2, KLF5, ESRRB, miR-291-3p, miR-294, miR-295, NR5A1, NR5A2, TBX3, MBD3sh, TH2A, TH2B, and P53DD. RNAs for these reprogramming factors are available from TriLink. It should be noted that gene symbols herein are denoted according to human nomenclature, but the use of uppercase or lowercase letters is not intended to limit the species. For example, the use of all uppercase letters does not exclude genes derived from mice or rats. However, in the Examples, the gene symbols are indicated in accordance with the actual species used.

The reprogramming factor may be RNA. The RNA may be mRNA. The RNA of the reprogramming factor may be modified with at least one selected from the group consisting of pseudouridine (Ψ), 5-methyluridine (5meU), N1-methylpseudouridine (melΨ), 5-methoxyuridine (5moU), 5-hydroxymethyluridine (5hmU), 5-formyluridine (5fU), 5-carboxymethyl ester uridine (5camU), thienoguanosine (thG), N4-methylcytidine (me4C), 5-methylcytidine (m5C), 5-methoxycytidine (5moC), 5-hydroxymethylcytidine (5hmC), 5-hydroxycytidine (5hoC), 5-formylcytidine (5fC), 5-carboxycytidine (5caC), N6-methyl-2-aminoadenosine (m6DAP), diaminopurine (DAP), 5-methyluridine (m5U), 2′-O-methyluridine (Um or m2′-OU), 2-thiouridine (s2U), and N6-methyladenosine (m6A). The RNA of the reprogramming factor may be polyadenylated.

The reprogramming factor may be introduced into a cell, for example, by a lipofection method. The lipofection method is a technique in which a complex of nucleic acid, which is negatively charged, and cationic lipid is formed through electrostatic interactions, and the complex is introduced into the cell via endocytosis or membrane fusion. Lipofection has advantages such as causing minimal damage to cells, high introduction efficiency, simple operation, and short processing time.

The reprogramming factor may be introduced into cells cultured, for example, using an RNA transfection reagent. For instance, when the cells are mononuclear cells, RNA may be introduced into the mononuclear cells immediately after isolation from blood.

Alternatively, the reprogramming factor may be introduced into the cells using a viral vector, for example. The viral vector may be an RNA viral vector. By using an RNA viral vector, it is possible to induce cells in an integration-free manner. The RNA viral vector may be a single-stranded positive-sense RNA virus. Alternatively, the RNA viral vector may be a single-stranded negative-sense RNA virus. The RNA viral vector may be a Sendai virus vector, a retrovirus vector, a lentivirus vector, or a rubella virus vector. The Sendai virus vector may be a temperature-sensitive Sendai virus vector in which the stability of the viral nucleic acid decreases at a predetermined temperature or higher. The viral nucleic acid of the temperature-sensitive Sendai virus vector is stable below the predetermined temperature. The viral nucleic acid may be viral DNA or viral RNA. The viral nucleic acid may be a viral genome. The reduction in the stability of the viral nucleic acid may refer to at least one of the degradation of the viral nucleic acid and suppression of replication or proliferation of the viral nucleic acid. When the stability of the viral nucleic acid is reduced, at least one of the proliferation, replication rate, and gene expression level of the viral nucleic acid is decreased. The predetermined temperature may be, for example, from 36.5° C. to 37.5° C., from 36.6° C. to 37.4° C., 36.7° C. or higher and 37.3° C. or lower, from 36.8° C. to 37.2° C., from 36.9° C. to 37.1° C., or 37° C. The stability of the viral nucleic acid of the temperature-sensitive Sendai virus vector—that is, at least one of proliferation, replication rate, and gene expression level—is high below the predetermined temperature and low at or above the predetermined temperature. For example, the proliferation rate or gene expression level of a temperature-sensitive Sendai virus vector in cells cultured at 37° C. is ½ or less, ⅓ or less, ⅕ or less, 1/10 or less, or 1/20 or less than that in cells cultured at 32° C.

After introducing the reprogramming factor into the cells and culturing the cells, all of the cells into which the reprogramming factor has been introduced are passaged at least once. Thereafter, all of the cells into which the reprogramming factor has been introduced may be passaged multiple times. All of the cells into which the reprogramming factor has been introduced may be passaged until stem cells are established.

Here, “passaging all of the cells into which the reprogramming factor has been introduced” refers, for example, to passaging the reprogrammed cells without distinguishing them based on gene expression status. For example, at the time of passaging, the cells into which the reprogramming factor has been introduced may be seeded in the same culture vessel without distinguishing them based on gene expression status. Alternatively, “passaging all of the cells into which the reprogramming factor has been introduced” refers, for example, to passaging the cells without distinguishing them based on the degree of reprogramming. For example, at the time of passaging, the cells into which the reprogramming factor has been introduced may be seeded in the same culture vessel without distinguishing them based on the degree of reprogramming.

Alternatively, “passaging all of the cells into which the reprogramming factor has been introduced” refers, for example, to passaging the cells without distinguishing them based on morphology. For example, at the time of passaging, the cells into which the reprogramming factor has been introduced may be seeded in the same culture vessel without distinguishing them based on morphology. Alternatively, “passaging all of the cells into which the reprogramming factor has been introduced” refers, for example, to passaging the cells without distinguishing them based on size. For example, at the time of passaging, the cells into which the reprogramming factor has been introduced may be seeded in the same culture vessel without distinguishing them based on size.

Alternatively, “passaging all of the cells into which the reprogramming factor has been introduced” refers to passaging the cells without performing cloning. For example, in the case of passaging without cloning, it is not necessary to pick up colonies formed by the cells into which the reprogramming factor has been introduced. For example, in the case of passaging without cloning, it is not necessary to isolate multiple colonies formed by the cells into which the reprogramming factor has been introduced from one another. For instance, at the time of passaging, cells forming multiple distinct colonies may be mixed together and seeded in the same culture vessel. Further, for example, in the case of passaging without cloning, it is not necessary to clone a single colony formed by the cells into which the reprogramming factor has been introduced. For example, at the time of passaging, multiple colonies may be mixed and seeded in the same culture vessel.

For example, when the cells into which the reprogramming factor has been introduced are cultured under adherent conditions, all of the adherent cells may be passaged. For instance, at the time of passaging, all of the cells detached from the culture vessel may be seeded in the same culture vessel. For example, all of the cells detached from the culture vessel using a detachment solution may be passaged. For example, cells that have not formed colonies may also be passaged. When the cells into which the reprogramming factor has been introduced are cultured under suspension conditions, all of the suspension-cultured cells may be passaged.

At the time of passaging the cells into which the reprogramming factor has been introduced, the cells are seeded at a low density in the medium or in the culture vessel. Herein, “low density” refers, for example, to a density of 1 cell/cm² or more and not more than 0.25×10⁴ cells/cm², not more than 1.25×10³ cells/cm², not more than 0.25×10³ cells/cm², not more than 0.25×10² cells/cm², or not more than 0.25×10¹ cells/cm². Alternatively, “low density” refers to a cell density at which 10 or fewer, 9 or fewer, 8 or fewer, 7 or fewer, 6 or fewer, 5 or fewer, 4 or fewer, 3 or fewer, or 2 or fewer cells are in contact with each other, and in which 11 or more cells are not in contact with one another. It is also acceptable that multiple cell aggregates formed by contact between 10 or fewer cells exist. Alternatively, “low density” may refer to a confluency, based on a state in which the entire bottom surface of a culture vessel is covered by cells defined as 100% confluent, of not more than 5%, not more than 4%, not more than 3%, not more than 2%, not more than 1%, not more than 0.5%, not more than 0.1%, not more than 0.05%, or not more than 0.01% confluency. Further alternatively, “low density” refers, for example, to a density at which individual seeded cells (single cells) do not contact one another. For instance, single cells may be seeded into wells of a well plate. The well plate may be, for example, a 12-well plate or a 96-well plate. According to the inventors' findings, by seeding the cells at low density into the medium during the passaging of cells into which the reprogramming factor has been introduced, it is possible to suppress the residual presence of Sendai virus in the pluripotent stem cells induced from the cells. In the induced pluripotent stem cells, the proportion of cells in which the Sendai virus remains is, for example, 4% or less, 3% or less, 2% or less, 1% or less, 0.5% or less, or 0%.

The cells according to any one of the first to twenty-first embodiments may be somatic cells differentiated from stem cells. Examples of somatic cells differentiated from stem cells include, but are not limited to, nerve cells, neural stem cells, retinal cells, corneal cells, cardiomyocytes, muscle cells, chondrocytes, immune cells, pancreatic β cells, fibroblasts, blood cells, dental pulp stem cells, keratinocytes, dermal papilla cells, oral epithelial cells, somatic stem/progenitor cells, and urinary cells. The blood cells may be nucleated cells, red blood cells, granulocytes, or platelets.

Nerve cells are suitable, for example, for transplantation into patients with Parkinson's disease. Retinal cells and corneal cells are suitable, for example, for transplantation into patients with ocular diseases. Cardiomyocytes are suitable, for example, for transplantation into patients with heart failure. Neural stem cells are suitable, for example, for transplantation into patients with spinal cord injury. Platelets are suitable, for example, for transfusion. Immune cells are suitable, for example, for transplantation into cancer patients. Pancreatic β cells are suitable, for example, for transplantation into patients with diabetes. Chondrocytes are suitable, for example, for transplantation into patients with joint disorders.

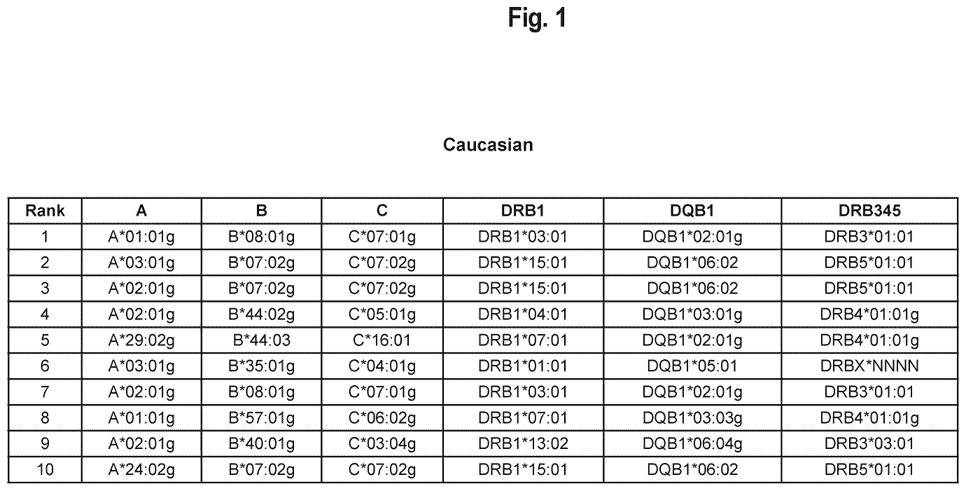

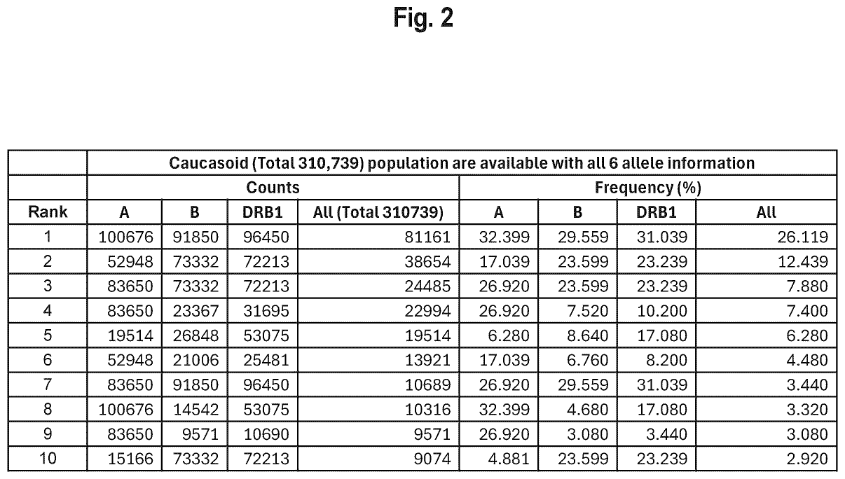

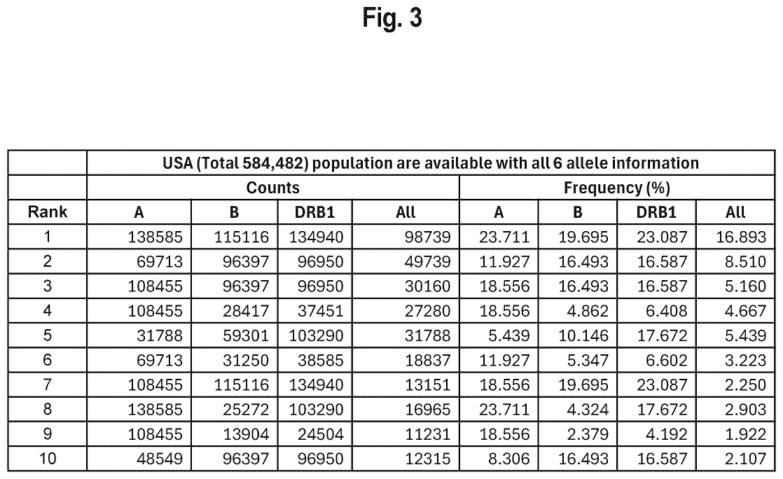

(Example 1: Compatibility rate of HLA homozygous donors in Caucasians) In the following, the allele frequencies were calculated using the database (http://www.allelefrequencies.net/hla6003a.asp), with the total number of all HLA types in Caucasians as the denominator. As shown in FIGS. 1, 2, and 3, cells from Donor 1, a Caucasian homozygous for each of HLA-A*01:01g, HLA-B*08:01g, HLA-C*07:01g, HLA-DRB1*03:01, HLA-DQB1*02:01g, and HLA-DRB3*01:01, were calculated to match 26.119% of 310,739 randomly selected Caucasians and 16.893% of 584,482 randomly selected individuals in the United States.

Cells from Donor 2, a Caucasian homozygous for each of HLA-A*03:01g, HLA-B*07:02g, HLA-C*07:02g, HLA-DRB1*15:01, HLA-DQB1*06:02, and HLA-DRB5*01:01, were calculated to match 12.439% of 310,739 randomly selected Caucasians and 8.510% of 584,482 randomly selected individuals in the United States.

Cells from Donor 3, a Caucasian homozygous for each of HLA-A*02:01g, HLA-B*07:02g, HLA-C*07:02g, HLA-DRB1*15:01, HLA-DQB1*06:02, and HLA-DRB5*01:01, were calculated to match 7.880% of 310,739 randomly selected Caucasians and 5.160% of 584,482 randomly selected individuals in the United States.

Cells from Donor 4, a Caucasian homozygous for each of HLA-A*02:01g, HLA-B*44:02g, HLA-C*05:01g, HLA-DRB1*04:01, HLA-DQB1*03:01g, and HLA-DRB4*01:01g, were calculated to match 7.400% of 310,739 randomly selected Caucasians and 4.667% of 584,482 randomly selected individuals in the United States.

Cells from Donor 5, a Caucasian homozygous for each of HLA-A*29:02g, HLA-B*44:03, HLA-C*16:01, HLA-DRB1*07:01, HLA-DQB1*02:01g, and HLA-DRB4*01:01g, were calculated to match 6.280% of 310,739 randomly selected Caucasians and 5.439% of 584,482 randomly selected individuals in the United States.

Cells from Donor 6, a Caucasian homozygous for each of HLA-A*03:01g, HLA-B*35:01g, HLA-C*04:01g, HLA-DRB1*01:01, HLA-DQB1*05:01, and HLA-DRBX*NNNN, were calculated to match 4.480% of 310,739 randomly selected Caucasians and 3.223% of 584,482 randomly selected individuals in the United States.

Cells from Donor 7, a Caucasian homozygous for each of HLA-A*02:01g, HLA-B*08:01g, HLA-C*07:01g, HLA-DRB1*03:01, HLA-DQB1*02:01g, and HLA-DRB3*01:01, were calculated to match 3.440% of 310,739 randomly selected Caucasians and 2.250% of 584,482 randomly selected individuals in the United States.

Cells from Donor 8, a Caucasian homozygous for each of HLA-A*01:01g, HLA-B*57:01g, HLA-C*06:02g, HLA-DRB1*07:01, HLA-DQB1*03:03g, and HLA-DRB4*01:01g, were calculated to match 3.320% of 310,739 randomly selected Caucasians and 2.903% of 584,482 randomly selected individuals in the United States.

Cells from Donor 9, a Caucasian homozygous for each of HLA-A*02:01g, HLA-B*40:01g, HLA-C*03:04g, HLA-DRB1*13:02, HLA-DQB1*06:04g, and HLA-DRB3*03:01, were calculated to match 3.080% of 310,739 randomly selected Caucasians and 1.922% of 584,482 randomly selected individuals in the United States.

Cells from Donor 10, a Caucasian homozygous for each of HLA-A*24:02g, HLA-B*07:02g, HLA-C*07:02g, HLA-DRB1*15:01, HLA-DQB1*06:02, and HLA-DRB5*01:01, were calculated to match 2.920% of 310,739 randomly selected Caucasians and 2.107% of 584,482 randomly selected individuals in the United States.

### Example 2: Production of iPS Cells Derived from a Caucasian Donor

A hematopoietic cell culture medium that was serum-free and free of animal-derived components (Stemspan ACF, STEMCELL TECHNOLOGIES) was supplemented with recombinant FLT3 ligand (PEPROTECH), recombinant human TPO (PEPROTECH), recombinant human IL-6 (PEPROTECH), recombinant human G-CSF (PEPROTECH), and recombinant human SCF (PEPROTECH) to prepare the hematopoietic cell medium. In each well of a 12-well dish, 2×10⁵ blood cells (peripheral blood mononuclear cells) derived from Caucasian Donor 1 described in Example 1 were seeded. The 12-well dish was then placed in a CO₂ incubator at 37° C. and cultured under suspension conditions to expand non-T cells.

Three days after initiation of cell culture, hematopoietic cell culture medium was additionally supplied as appropriate to each well. On day six of culture, Oct3/4, SOX2, KLF4, and c-Myc were introduced into the cells in each well. Thereafter, hematopoietic cell culture medium was added to each well, and the cells were cultured on a laminin 511-coated dish, which served as a stem cell culture substrate. Starting from day two post-infection, the culture medium in each well was half-exchanged with stem cell culture medium every other day. On day fourteen post-infection, the induction of ten or more iPS cell colonies was confirmed. iPS cells were also induced from the blood cells of each of Caucasian Donors 2, 3, 4, 5, 6, 7, 8, 9, and 10 described in Example 1, using a similar method.

### Example 3: Compatibility Rate of HLA-Homozygous Donors in Japanese Population

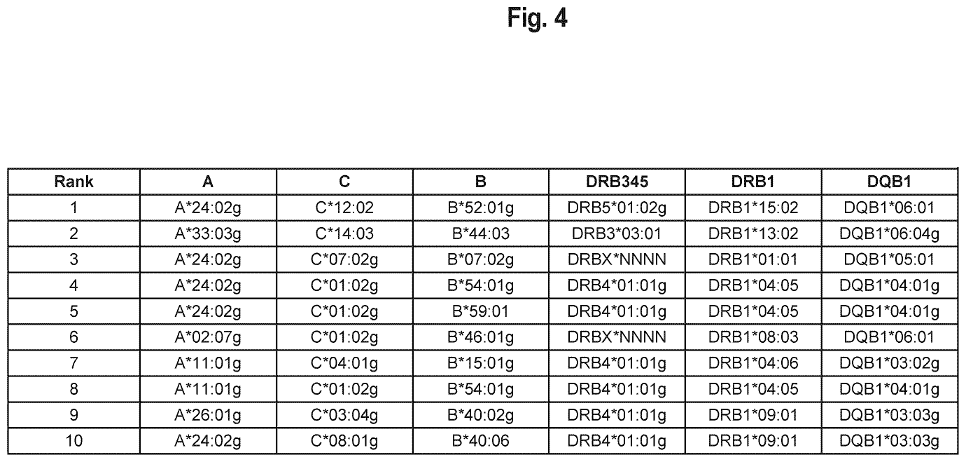

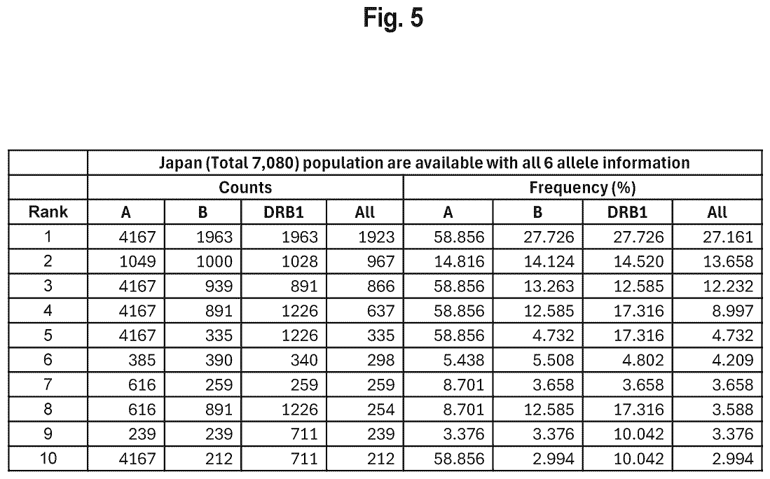

In the following, allele frequencies were calculated using the database (http://www.allelefrequencies.net/hla6003a.asp), based on the total number of HLA types in the Japanese population as the denominator. As shown in FIG. 4 and FIG. 5, cells from Japanese Donor 1, homozygous for each of HLA-A*24:02g, HLA-C*12:02, HLA-B*52:01g, HLA-DRB5*01:02g, HLA-DRB1*15:02, and HLA-DQB1*06:01, were calculated to match 27.161% of 7,080 randomly selected Japanese individuals.

Cells from Japanese Donor 2, homozygous for each of HLA-A*33:03g, HLA-C*14:03, HLA-B*44:03, HLA-DRB3*03:01, HLA-DRB1*13:02, and HLA-DQB1*06:04g, were calculated to match 13.658% of 7,080 randomly selected Japanese individuals.

Cells from Japanese Donor 3, homozygous for each of HLA-A*24:02g, HLA-C*07:02g, HLA-B*07:02g, HLA-DRBX*NNNN, HLA-DRB1*01:01, and HLA-DQB1*05:01, were calculated to match 12.232% of 7,080 randomly selected Japanese individuals.

Cells from Japanese Donor 4, homozygous for each of HLA-A*24:02g, HLA-C*01:02g, HLA-B*54:01g, HLA-DRB4*01:01g, HLA-DRB1*04:05, and HLA-DQB1*04:01g, were calculated to match 8.997% of 7,080 randomly selected Japanese individuals.

Cells from Japanese Donor 5, homozygous for each of HLA-A*24:02g, HLA-C*01:02g, HLA-B*59:01, HLA-DRB4*01:01g, HLA-DRB1*04:05, and HLA-DQB1*04:01g, were calculated to match 4.732% of 7,080 randomly selected Japanese individuals.

Cells from Japanese Donor 6, homozygous for each of HLA-A*02:07g, HLA-C*01:02g, HLA-B*46:01g, HLA-DRBX*NNNN, HLA-DRB1*08:03, and HLA-DQB1*06:01, were calculated to match 4.209% of 7,080 randomly selected Japanese individuals.

Cells from Japanese Donor 7, homozygous for each of HLA-A*11:01g, HLA-C*04:01g, HLA-B*15:01g, HLA-DRB4*01:01g, HLA-DRB1*04:06, and HLA-DQB103:02g, were calculated to match 3.658% of 7,080 randomly selected Japanese individuals.

Cells from Japanese Donor 8, homozygous for each of HLA-A*11:01g, HLA-C*01:02g, HLA-B*54:01g, HLA-DRB4*01:01g, HLA-DRB1*04:05, and HLA-DQB1*04:01g, were calculated to match 3.588% of 7,080 randomly selected Japanese individuals.

Cells from Japanese Donor 9, homozygous for each of HLA-A*26:01g, HLA-C*03:04g, HLA-B*40:02g, HLA-DRB4*01:01g, HLA-DRB1*09:01, and HLA-DQB1*03:03g, were calculated to match 3.376% of 7,080 randomly selected Japanese individuals.

Cells from Japanese Donor 10, homozygous for each of HLA-A*24:02g, HLA-C*08:01g, HLA-B*40:06, HLA-DRB4*01:01g, HLA-DRB1*09:01, and HLA-DQB1*03:03g, were calculated to match 2.994% of 7,080 randomly selected Japanese individuals.

### Example 4: Generation of iPS Cells Derived from Japanese Donors

iPS cells were induced from blood cells of each of Japanese Donors 1 to 10 described in Example 3 using the same method as in Example 2.

### Example 5: HLA Homozygous Donor Matching Rate in the U.S. Population

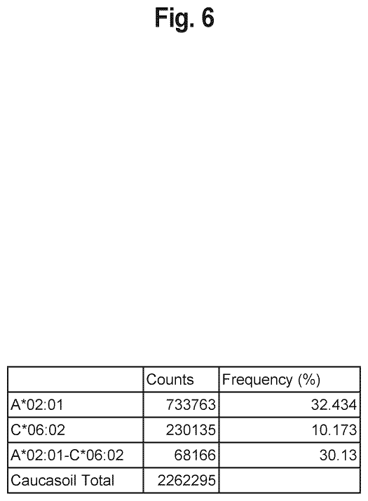

Allele frequencies were calculated using the database (http://www.allelefrequencies.net/hla6003a.asp), with all HLA types in the U.S. population used as the denominator. Cells from a U.S. donor homozygous for HLA-A*02:01 were calculated to match 35.9% of randomly selected individuals in the U.S. population. Cells from a U.S. donor homozygous for HLA-C*06:02 were calculated to match 61.0% of randomly selected individuals in the U.S. population. As shown in FIG. 6, cells from a U.S. donor homozygous for HLA-A*02:01 were calculated to match 32.434% of randomly selected Caucasians. Cells from a U.S. donor homozygous for HLA-C*06:02 were calculated to match 10.173% of randomly selected Caucasians. Cells from a U.S. donor homozygous for both HLA-A*02:01 and HLA-C*06:02 were calculated to match 30.13% of randomly selected Caucasians.

### Example 6: Generation of iPS Cells Derived from U.S. Donors

iPS cells were induced from blood cells of a U.S. donor homozygous for each of HLA-A*02:01, HLA-C*06:02, and HLA-DPA1*01:03 using the same method as in Example 2.

1. A cell that is homozygous for at least one, two, three, four, or all of the group consisting of HLA-A*01:01g, HLA-B*08:01g, HLA-DRB1*03:01, HLA-C*07:01g, HLA-DQB1*02:01g, and HLA-DRB3*01:01.

2. A cell that is homozygous for at least one, two, three, four, or all of the group consisting of HLA-A*03:01g, HLA-B*07:02g, HLA-DRB1*15:01, HLA-C*07:02g, HLA-DQB1*06:02, and HLA-DRB5*01:01.

3. A cell that is homozygous for at least one, two, three, four, or all of the group consisting of HLA-A*02:01g, HLA-B*07:02g, HLA-DRB1*15:01, HLA-C*07:02g, HLA-DQB1*06:02, and HLA-DRB5*01:01.

4. A cell that is homozygous for at least one, two, three, four, or all of the group consisting of HLA-A*02:01g, HLA-B*44:02g, HLA-DRB1*04:01, HLA-C*05:01g, HLA-DQB1*03:01g, and HLA-DRB4*01:01g.

5. A cell that is homozygous for at least one, two, three, four, or all of the group consisting of HLA-A*29:02g, HLA-B*44:03, HLA-DRB1*07:01, HLA-C*16:01, HLA-DQB1*02:01g, and HLA-DRB4*01:01g.

6. A cell that is homozygous for at least one, two, three, four, or all of the group consisting of HLA-A*03:01g, HLA-B*35:01g, HLA-C*04:01g, HLA-DRB1*01:01, HLA-DQB1*05:01, and HLA-DRBX*NNNN.

7. A cell that is homozygous for at least one, two, three, four, or all of the group consisting of HLA-A*02:01g, HLA-B*08:01g, HLA-C*07:01g, HLA-DRB1*03:01, HLA-DQB1*02:01g, and HLA-DRB3*01:01.

8. A cell that is homozygous for at least one, two, three, four, or all of the group consisting of HLA-A*01:01g, HLA-B*57:01g, HLA-C*06:02g, HLA-DRB1*07:01, HLA-DQB1*03:03g, and HLA-DRB4*01:01g.

9. A cell that is homozygous for at least one, two, three, four, or all of the group consisting of HLA-A*02:01g, HLA-B*40:01g, HLA-C*03:04g, HLA-DRB1*13:02, HLA-DQB1*06:04g, and HLA-DRB3*03:01.

10. A cell that is homozygous for at least one, two, three, four, or all of the group consisting of HLA-A*24:02g, HLA-B*07:02g, HLA-C*07:02g, HLA-DRB1*15:01, HLA-DQB1*06:02, and HLA-DRB5*01:01.

11. A cell that is homozygous for at least one, two, three, four, or all of the group consisting of HLA-A*24:02g, HLA-B*52:01g, HLA-DRB1*15:02, HLA-C*12:02, HLA-DRB5*01:02g, and HLA-DQB1*06:01.

12. A cell that is homozygous for at least one, two, three, four, or all of the group consisting of HLA-A*33:03g, HLA-B*44:03, HLA-DRB1*13:02, HLA-C*14:03, HLA-DRB3*03:01, and HLA-DQB1*06:04g.

13. A cell that is homozygous for at least one, two, three, four, or all of the group consisting of HLA-A*24:02g, HLA-B*07:02g, HLA-DRB1*01:01, HLA-C*07:02g, HLA-DRBX*NNNN, and HLA-DQB1*05:01.

14. A cell that is homozygous for at least one, two, three, four, or all of the group consisting of HLA-A*24:02g, HLA-B*54:01g, HLA-DRB1*04:05, HLA-C*01:02g, HLA-DRB4*01:01g, and HLA-DQB1*04:01g.

15. A cell that is homozygous for at least one, two, three, four, or all of the group consisting of HLA-A*24:02g, HLA-B*59:01, HLA-DRB1*04:05, HLA-C*01:02g, HLA-DRB4*01:01g, and HLA-DQB1*04:01g.

16. A cell that is homozygous for at least one, two, three, four, or all of the group consisting of HLA-A*02:07g, HLA-C*01:02g, HLA-B*46:01g, HLA-DRBX*NNNN, HLA-DRB1*08:03, and HLA-DQB1*06:01.

17. A cell that is homozygous for at least one, two, three, four, or all of the group consisting of HLA-A*11:01g, HLA-C*04:01g, HLA-B*15:01g, HLA-DRB4*01:01g, HLA-DRB1*04:06, and HLA-DQB103:02g.

18. A cell that is homozygous for at least one, two, three, four, or all of the group consisting of HLA-A*11:01g, HLA-C*01:02g, HLA-B*54:01g, HLA-DRB4*01:01g, HLA-DRB1*04:05, and HLA-DQB1*04:01g.

19. A cell that is homozygous for at least one, two, three, four, or all of the group consisting of HLA-A*26:01g, HLA-C*03:04g, HLA-B*40:02g, HLA-DRB4*01:01g, HLA-DRB1*09:01, and HLA-DQB1*03:03g.

20. A cell that is homozygous for at least one, two, three, four, or all of the group consisting of HLA-A*24:02g, HLA-C*08:01g HLA-B*40:06, HLA-DRB4*01:01g, HLA-DRB1*09:01, and HLA-DQB1*03:03g.

21. A cell that is homozygous for at least one, two, or all of the group consisting of HLA-A*02:01, HLA-C*06:02, and HLA-DPA1*01:03.
